(19) United States
(12) Patent Application Publication
(10) Pub. No.: US 2016/0213443 A1
(43) Pub. Date: Jul. 28, 2016

(54) Surgical Retraction Frame, Surgical Retraction Systems, and Methods of Using Same

(71) Applicants: Southern Research Institute, Birmingham, AL (US); The UAB Research Foundation, Birmingham, AL (US)

(72) Inventors: JASON LUECK, Hoover, AL (US); JASON A. LOWE, Homewood, AL (US); ALAN W. EBERHARDT, Birmingham, AL (US)

(21) Appl. No.: 15/004,112

(22) Filed: Jan. 22, 2016

Related U.S. Application Data
(60) Provisional application No. 62/106,416, filed on Jan. 22, 2015.

(51) Int. Cl. A61B 90/50; A61B 17/02; A61B 90/57; A61G 13/10

(52) U.S. Cl. CPC A61B 90/50; A61G 13/101; A61B 17/0293; A61B 90/57; A61B 2090/571

(57) ABSTRACT

A frame for supporting one or more surgical tools during a surgical procedure. The frame is connectable to a surgical table and has at least one transverse support assembly and at least one longitudinal support assembly. Each longitudinal support assembly extends between and is coupled to the first transverse support assembly and either a second transverse support assembly or a support post that is spaced from the first transverse support assembly relative to a longitudinal axis of the table. Each longitudinal support assembly has a shaft and at least one connector that is coupled to the shaft and configured to engage and support a surgical tool. The frame is connectable to the surgical table such that the first and second transverse support assemblies and the at least one longitudinal support assembly are spaced from the surgical table in an upward direction.

### CROSS-REFERENCE TO RELATED PATENT APPLICATION

This application claims priority to and the benefit of the filing date of U.S. Provisional Patent Application No. 62/106,416, filed Jan. 22, 2015, which application is incorporated herein by reference in its entirety.

### FIELD

This invention relates to frames for supporting surgical tools during surgical procedures and, more particularly, to frames for supporting surgical retraction tools during surgical retraction procedures.

### JOINT RESEARCH AGREEMENT

The presently claimed invention was made by or on behalf of the below listed parties to a joint research agreement. The joint research agreement was in effect on or before the earliest effective filing date of the claimed invention, and the claimed invention was made as a result of activities undertaken within the scope of the joint research agreement. The parties to the joint research agreement are (1) the Board of Trustees of the University of Alabama for the University of Alabama at Birmingham, (2) the UAB Research Foundation, and Southern Research Institute.

### BACKGROUND

Pelvic fractures are a common orthopedic trauma seen in operating rooms. Typically, to treat such pelvic fractures, a surgeon affixes support plates to the pelvis to thereby allow the fracture to heal properly. However, gaining physical access to the injury site is a difficult process, requiring the surgeon to move many soft tissues (muscles, nerves, arteries, veins, etc.) by several inches. Compounding this problem is that some tissues cannot be moved without causing significant damage. Consequently, the surgeon must move back and forth between several surgical windows to repair the trauma.

Physical limitations prohibit using more than one surgical window at any given time, that is, the surgeon must remove hardware from one window before working in the next. Additionally, the surgeon must remove radio-opaque materials each time an X-ray is taken, adding significant time to a long surgical procedure. Appropriate access to the injury site requires the use of a large surgical team, including the surgeon and at least two assistants, with each assistant using two retractors. This creates a surgical environment with many hands and bodies in a small place, making it difficult to perform the surgery.

Existing surgical retraction systems, such as the Bookwalter-style retraction system 1 depicted in FIG. 1, are not designed to work in multiple surgical windows, and the set-up/take-down time for these systems complicates their use in pelvic surgeries. In fact, these conventional surgical retraction systems are designed for other surgical procedures and are not optimized for orthopedic applications.

Thus, there is a need for devices, systems, and methods that allow surgeons to quickly move back and forth between surgical windows during surgical retraction procedures. There is a further need for devices, systems, and methods that permit initiation of X-ray imaging of a patient without requiring removal of significant portions of the surgical retraction frame. There is still a further need for devices, systems, and methods that will (a) reduce the time required to complete surgical retraction procedures and (b) reduce the number of assistants required to complete surgical retraction procedures.

### SUMMARY

Described herein is a frame for supporting one or more surgical tools during a surgical procedure. The frame is connectable to a surgical table having a longitudinal axis, a transverse axis that is substantially perpendicular to the longitudinal axis, a first end, an opposed second end spaced apart from the first end relative to the longitudinal axis, a proximal side extending from the first end to the second end, and an opposed distal side extending from the first end to the second end and spaced apart from the proximal side relative to the transverse axis. The frame can have first and second transverse support assemblies and at least one longitudinal support assembly. Each transverse support assembly can be configured to extend from the proximal side of the table to the distal side of the table and have a shaft with a central axis. The second transverse support assembly can be spaced from the first transverse support assembly relative to the longitudinal axis of the table. The at least one longitudinal support assembly can extend between and be operatively coupled to the first and second transverse support assemblies. The at least one longitudinal support assembly can comprise a shaft and at least one connector securely coupled to the shaft and configured to engage and support a surgical tool. The frame can be connectable to the surgical table such that the first and second transverse support assemblies and the at least one longitudinal support assembly are spaced from the surgical table in an upward direction relative to a vertical axis that is substantially perpendicular to both the longitudinal axis and the transverse axis of the table. Optionally, the at least one longitudinal support assembly can be radiolucent. Optionally, any connectors coupled to the at least one longitudinal support assembly can also be radiolucent. Optionally, the first and second transverse support assemblies and the at least one longitudinal support assembly can be radiolucent. Optionally, the entire frame can be radiolucent.

The frame can also have at least first and second pairs of opposed support posts. Each support post can have a first end and an opposed second end. The first end of each support post can be operatively connectable to the table. Each support post can extend upwardly from the first end of the support post such that the second end of the support post is spaced from the first end of the support post relative to the vertical axis. Each pair of opposed support posts can have a proximal support post and a distal support post that are spaced apart relative to the transverse axis of the table. The proximal support post can be operatively connectable to the proximal side of the table, and the distal support post can be operatively connectable to the distal side of the table. The first pair of opposed support posts can be operatively coupled to and configured to support the first transverse support assembly, and the second pair of opposed support posts can be operatively coupled to and configured to support the second transverse support assembly.

Also described herein is a radiolucent frame for supporting one or more surgical tools during a surgical procedure. The frame can be connectable to a surgical table. The frame can have at least one support assembly. The at least one support assembly can have at least one connector configured to engage and support a surgical tool. The frame can be connectable to the surgical table such that the at least one support assembly is spaced from the surgical table in an upward direction relative to a vertical axis. At least one of the support assemblies can be radiolucent. Optionally, at least one connector of a radiolucent support assembly can also be radiolucent.

Also described herein is a frame for supporting one or more surgical tools during a surgical procedure. The frame can be connectable to a surgical table having a longitudinal axis, a transverse axis that is substantially perpendicular to the longitudinal axis, a first end, an opposed second end spaced apart from the first end relative to the longitudinal axis, a proximal side extending from the first end to the second end, and an opposed distal side extending from the first end to the second end and spaced apart from the proximal side relative to the transverse axis. The frame can have a first transverse support assembly, a plurality of support posts, and at least one longitudinal support assembly. The first transverse support assembly can be configured to extend from the proximal side of the table to the distal side of the table and have a shaft with a central axis. The plurality of support posts can include at least three support posts. Each support post can extend upwardly from the first end of the support post such that the second end of the support post is spaced from the first end of the support post relative to a vertical axis that is substantially perpendicular to both the longitudinal axis and the transverse axis of the table. The at least three support posts can include a pair of opposed support posts and at least a third support post. The pair of opposed support posts can have a proximal support post and a distal support post that are spaced apart relative to the transverse axis of the table. The first end of the proximal support post can be operatively connectable to the proximal side of the table and the first end of the distal support post can be operatively connectable to the distal side of the table. The pair of opposed support posts can be operatively coupled to and configured to support the first transverse support assembly. The third support post can be spaced from the pair of opposed support posts relative to the longitudinal axis of the table. The first end of the third support post can be secured to the proximal or distal side of the table. Alternatively, the first end of the third support post can be secured to the table at a position between the proximal and distal sides of the table relative to the transverse axis of the table. The at least one longitudinal support assembly can extend between and be operatively coupled to the first transverse support assembly and the third support post. The at least one longitudinal support assembly have a shaft and at least one connector securely coupled to the shaft and configured to engage and support a surgical tool. The frame can be connectable to the surgical table such that the first support assembly and the at least one longitudinal support assembly are spaced from the surgical table in an upward direction relative to the vertical axis.

Further described herein is a surgical retraction system including a frame as disclosed herein and at least one surgical tool that is secured to and supported by a connector of the frame. The at least one surgical tool can comprise a surgical retractor.

Additionally, described herein is a method of using one or more surgical tools. The method can include securing at least one surgical tool to a connector of a frame as disclosed herein. The method can also include selectively activating the at least one surgical tool.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DESCRIPTION OF THE FIGURES

These and other features of the preferred embodiments of the invention will become more apparent in the detailed description in which reference is made to the appended drawings wherein:

FIG. 1 is a perspective view of a conventional retraction system as is known in the art.

FIG. 2 is a perspective view of an exemplary surgical retraction system having a frame with first and second transverse support assemblies as disclosed herein.

FIG. 3 is a perspective view of an exemplary frame for supporting one or more surgical tools during a surgical procedure, such as, for example and without limitation, a surgical retraction procedure, as disclosed herein. As shown, the frame can have first and second transverse support assemblies as disclosed herein.

FIG. 4 is a cross-sectional side view of an exemplary longitudinal support assembly as disclosed herein.

FIG. 5A is a bottom perspective view of an exemplary longitudinal support assembly that is coupled to first and second transverse support assemblies as disclosed herein. FIG. 5B is a close-up view showing an exemplary connection between an end portion of the longitudinal support assembly and a shaft of a transverse support assembly.

FIG. 6A is a bottom perspective view of an exemplary longitudinal support assembly that is coupled to first and second transverse support assemblies as disclosed herein. As shown, a central axis of the longitudinal support assembly can be angularly oriented relative to a longitudinal axis of a surgical table. FIG. 6B is a close-up view showing an exemplary connection between an end portion of the longitudinal support assembly and a shaft of a transverse support assembly.

FIG. 7A is a side view of an exemplary retraction system as disclosed herein, showing selective adjustment of the height of first and second transverse support assemblies. FIG. 7B is a top view of an exemplary surgical retraction system as disclosed herein, showing selective adjustment of the position of the first and second transverse support assemblies relative to a longitudinal axis of a surgical table. FIG. 7C is an end perspective view of an exemplary retraction system as disclosed herein, showing selective adjustment of respective ends of first and second transverse support assemblies relative to a vertical axis.

FIGS. 8A-8C schematically depict exemplary force vectors experienced by a frame during a surgical retraction procedure as disclosed herein.

FIG. 9 is a perspective view of an exemplary fixation assembly as disclosed herein.

FIG. 10A is a perspective view of an exemplary connector as disclosed herein, and FIG. 10B is a side perspective view of the connector of FIG. 10A.

FIG. 11 is a perspective view of an exemplary surgical retraction system having a frame with a first transverse support assembly that is supported by first and second support posts and a third support post that is axially spaced from the first transverse support assembly and first and second support posts as disclosed herein.

FIG. 12A is a perspective view of an exemplary connector as disclosed herein, and FIG. 12B and FIG. 12C are side and perspective views of the connector of FIG. 12A.

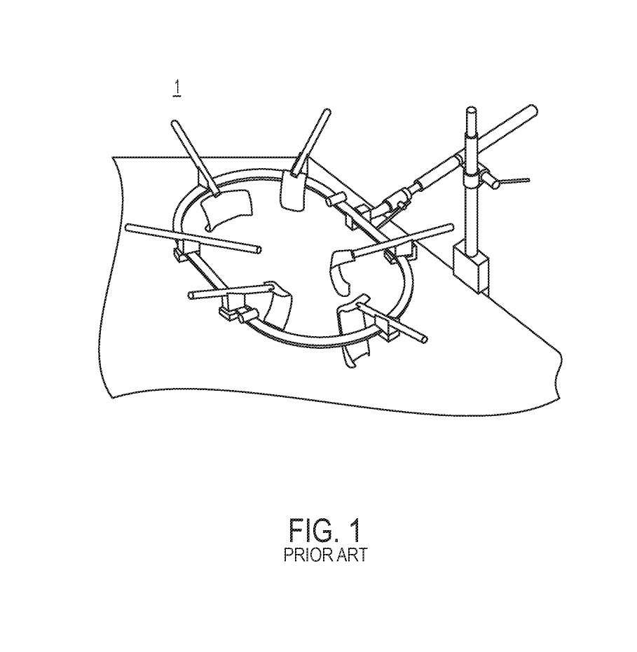

### DETAILED DESCRIPTION

The present invention can be understood more readily by reference to the following detailed description, examples, drawings, and claims, and their previous and following description. However, before the present devices, systems, and/or methods are disclosed and described, it is to be understood that this invention is not limited to the specific devices, systems, and/or methods disclosed unless otherwise specified, as such can, of course, vary. It is also to be understood that the terminology used herein is for the purpose of describing particular aspects only and is not intended to be limiting.

The following description of the invention is provided as an enabling teaching of the invention in its best, currently known embodiment. To this end, those skilled in the relevant art will recognize and appreciate that many changes can be made to the various aspects of the invention described herein, while still obtaining the beneficial results of the present invention. It will also be apparent that some of the desired benefits of the present invention can be obtained by selecting some of the features of the present invention without utilizing other features. Accordingly, those who work in the art will recognize that many modifications and adaptations to the present invention are possible and can even be desirable in certain circumstances and are a part of the present invention. Thus, the following description is provided as illustrative of the principles of the present invention and not in limitation thereof.

As used throughout, the singular forms “a,” “an” and “the” comprise plural referents unless the context clearly dictates otherwise. Thus, for example, reference to “a connector” can comprise two or more such connectors unless the context indicates otherwise.

Ranges can be expressed herein as from “about” one particular value, and/or to “about” another particular value. When such a range is expressed, another aspect comprises from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

As used herein, the terms “optional” or “optionally” mean that the subsequently described event or circumstance can or cannot occur, and that the description comprises instances where said event or circumstance occurs and instances where it does not.

The word “or” as used herein means any one member of a particular list and also comprises any combination of members of that list.

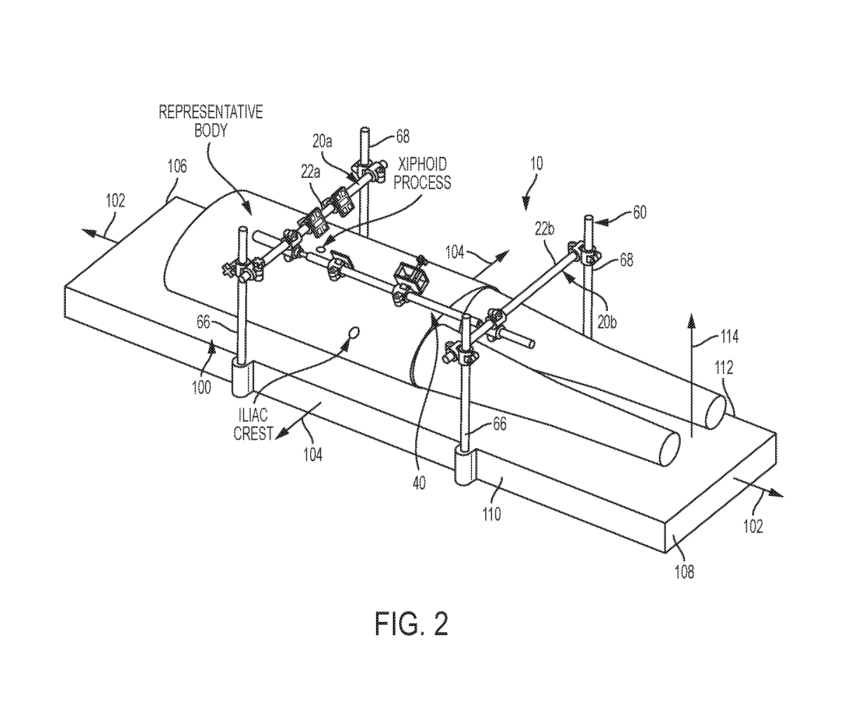

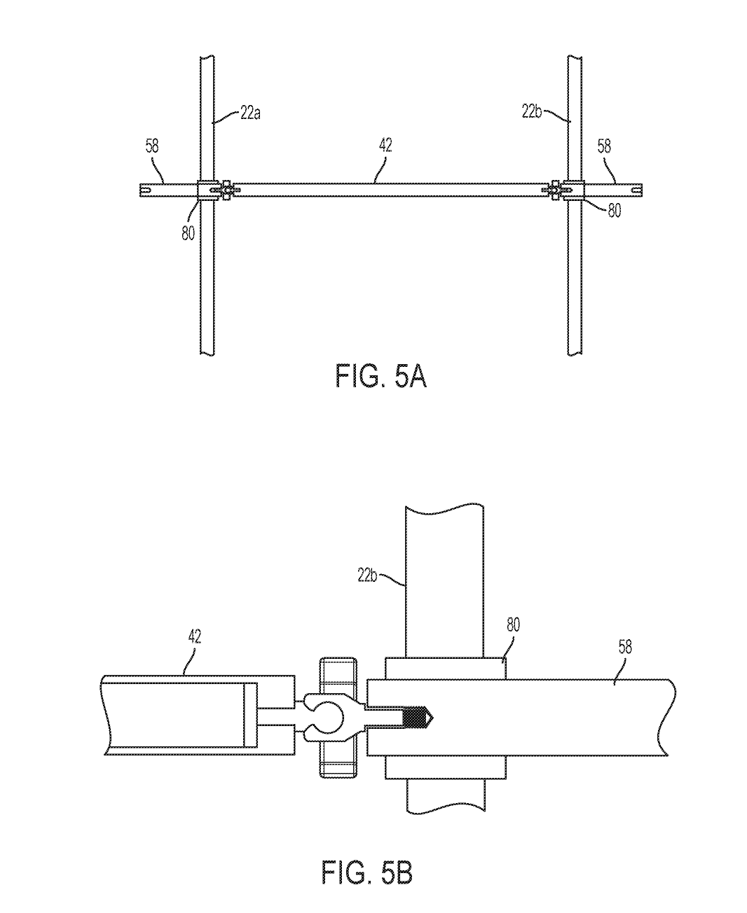

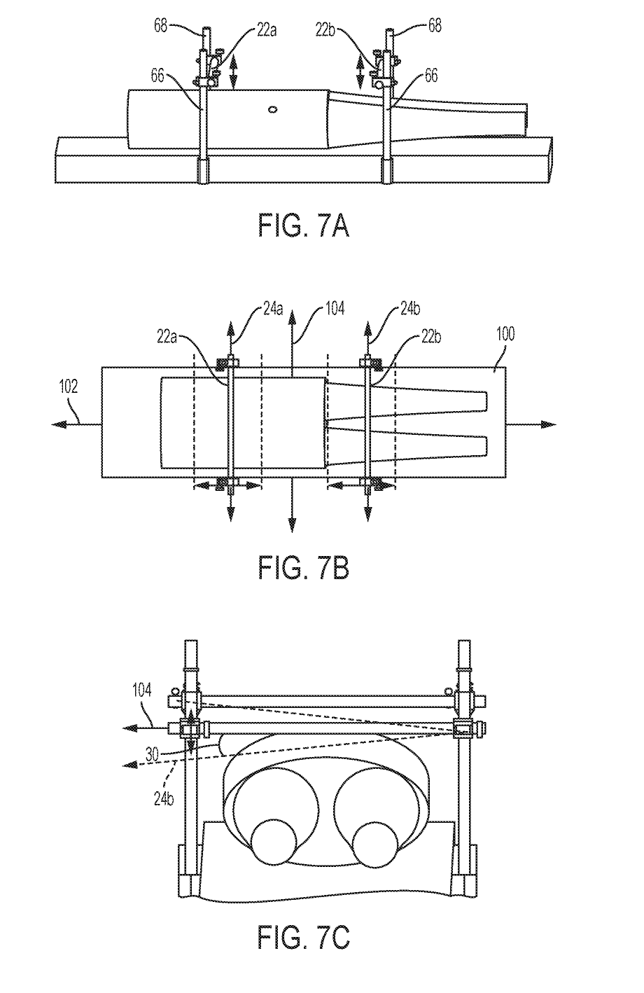

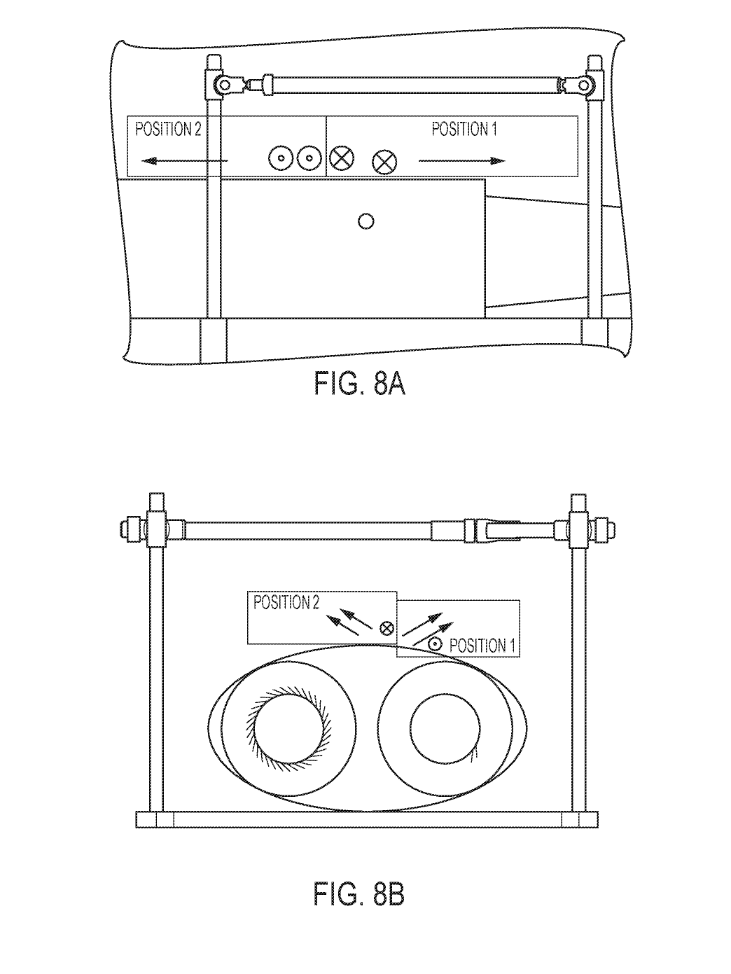

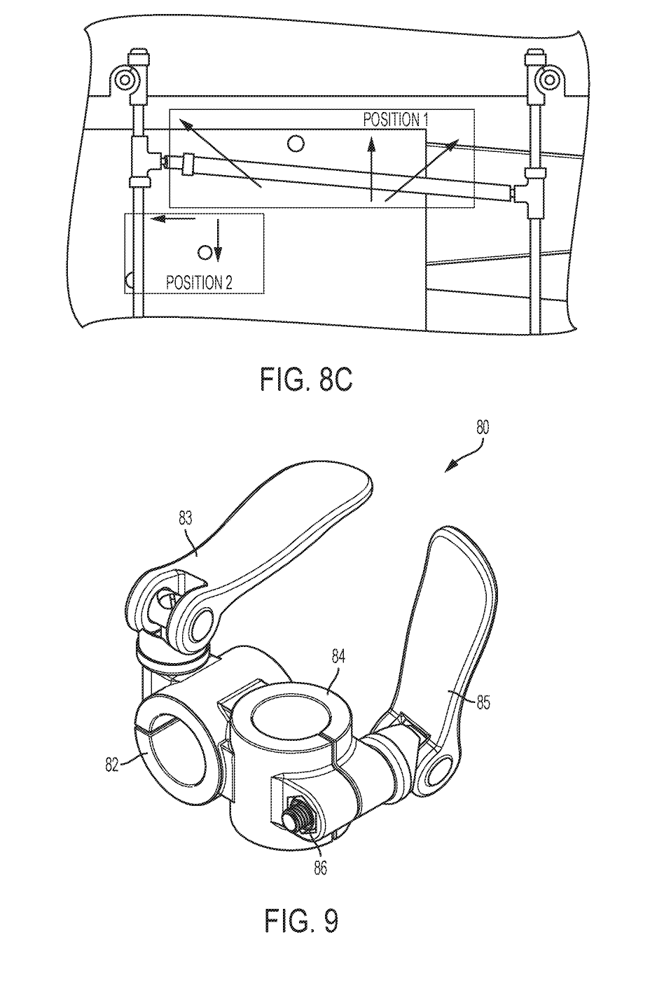

Described herein with reference to FIGS. 2-12C is a frame 10 for supporting one or more surgical tools during a surgical procedure, such as, for example and without limitation, a surgical retraction procedure. As further disclosed herein, it is contemplated that the frame 10 can be configured to provide a rigid, high-strength structure that allows for quick adjustments in the position of surgical tools relative to multiple axes to accommodate any orientations or retraction directions required during the surgical procedure. This capability can reduce the time required to complete the surgical procedure by allowing a surgeon to quickly shift between surgical windows (e.g., incisions), or, more generally, to adjust within a surgical field, without the need for disassembling the frame. It is further contemplated that the frame 10 can be configured to reduce the need for assistants to hold surgical retractors or other surgical tools during the surgical procedure. In contrast, conventional static tool frames are unable to accommodate different force directions, and many adjustments are required to move these frames from a first orientation to a second orientation.

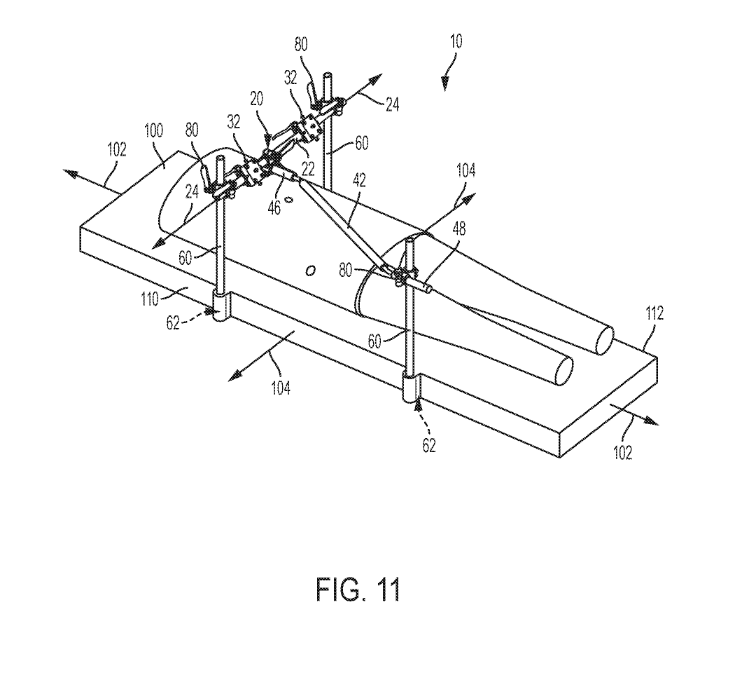

In exemplary aspects, as shown in FIGS. 2 and 11, the frame 10 can be connectable to a surgical table 100. In these aspects, it is contemplated that the surgical table 100 can be any structure that supports a subject/patient during a surgical procedure, such as, for example and without limitation, a conventional surgical table as is known in the art. However, it is further contemplated that the frame 10 can be used with any other base structure that secures the frame in an operative position relative to the subject/patient. It is contemplated that the surgical table 100 can have a longitudinal axis 102 and a transverse axis 104 that is substantially perpendicular to the longitudinal axis. It is further contemplated that the surgical table 100 can have a first end 106 and an opposed second end 108 spaced apart from the first end relative to the longitudinal axis 102. It is still further contemplated that the surgical table 100 can have a proximal side 110 extending from the first end 106 to the second end 108, and an opposed distal side 112 extending from the first end to the second end and spaced apart from the proximal side relative to the transverse axis 104.

### Frames Having First and Second Transverse Support Assemblies

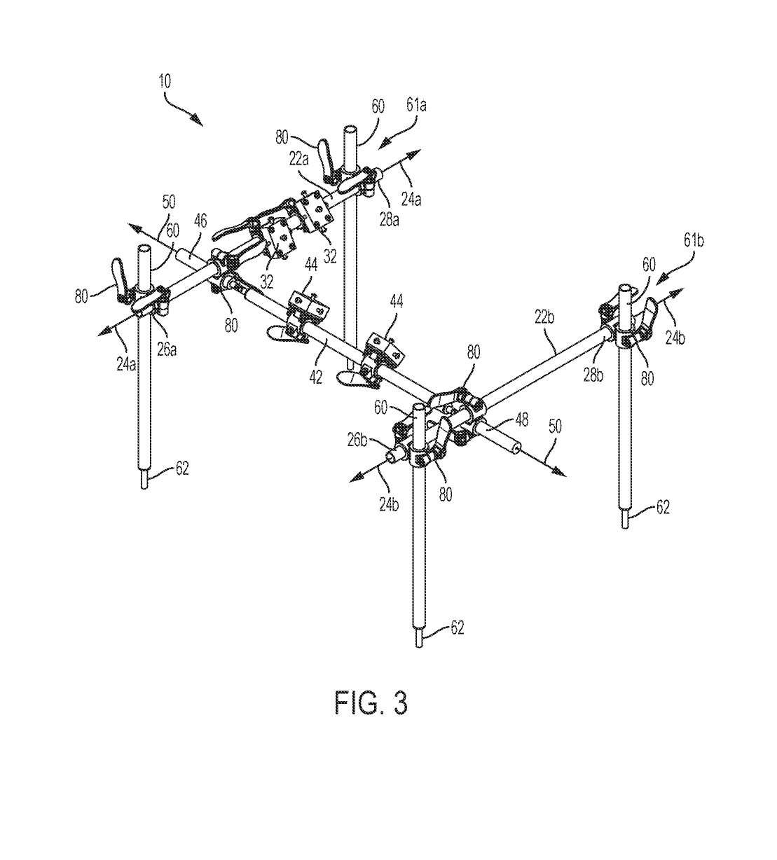

In one aspect, as depicted in FIGS. 2-3, the frame 10 can comprise at least first and second transverse support assemblies 20a, 20b. In this aspect, each transverse support assembly 20a, 20b can be configured to extend from the proximal side 110 of the table 100 to the distal side 112 of the table and can have a shaft 22a, 22b with a central axis 24a, 24b. As shown in FIGS. 2-3, 6A, and 7A-7B, the second transverse support assembly 20b can be spaced from the first transverse support assembly 20a relative to the longitudinal axis 102 of the table 100. In an additional aspect, at least one of the first and second transverse support assemblies 20a, 20b can further comprise at least one connector 32 securely coupled to the shaft 22a, 22b of the transverse support assembly 20a, 20b and configured to engage and support a surgical tool.

In another aspect, as shown in FIGS. 2-3, the frame 10 can comprise at least one longitudinal support assembly 40 extending between and operatively coupled to the first and second transverse support assemblies 20a, 20b. In this aspect, each longitudinal support assembly can comprise a shaft 42 and at least one connector 44 securely coupled to the shaft and configured to engage and support a surgical tool.

In exemplary aspects, as shown in FIG. 2, the frame 10 can be connectable to the surgical table 100 such that the first and second transverse support assemblies 20a, 20b and the at least one longitudinal support assembly 40 are spaced from the surgical table in an upward direction relative to a vertical axis 114 that is substantially perpendicular to both the longitudinal axis 102 and the transverse axis 104 of the table 100.

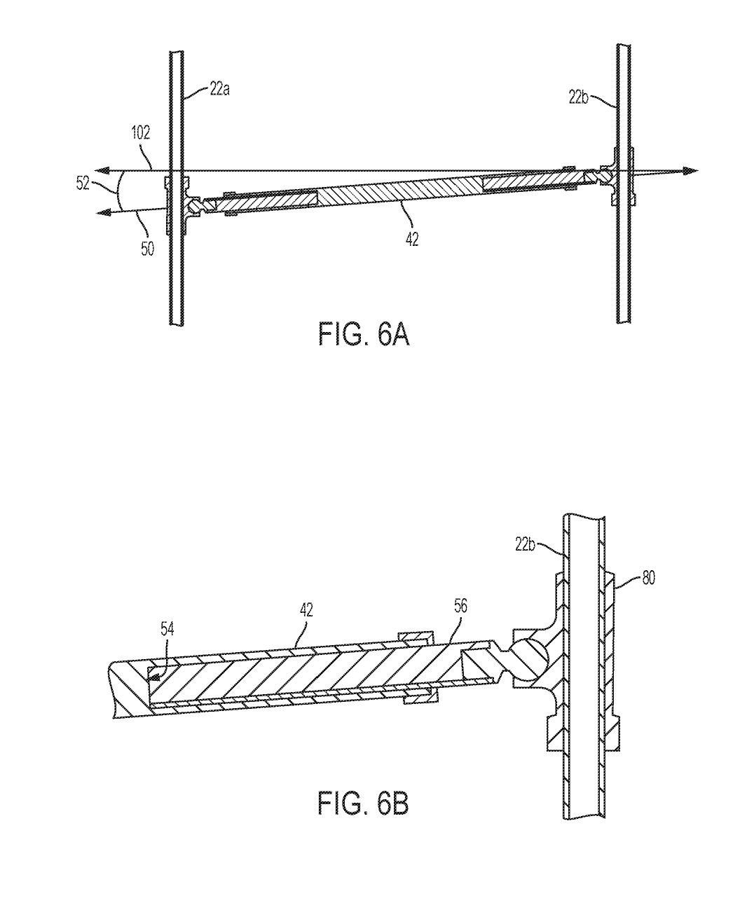

In a further aspect, and as depicted in FIG. 3, each longitudinal support assembly 40 of the at least one longitudinal support assembly can have a first end portion 46 operatively coupled to the shaft 22a of the first transverse support assembly 20a and a second end portion 48 operatively coupled to the shaft 22b of the second transverse support assembly 20b. In this aspect, and with reference to FIGS. 3 and 6A, the first end portion 46 of the longitudinal support assembly 40 can be selectively axially moveable relative to the central axis 24a of the shaft 22a of the first transverse support assembly 20a, and the second end portion 48 of the longitudinal support assembly 40 can be selectively axially moveable relative to the central axis 24b of the shaft 22b of the second transverse support assembly 20b.

Optionally, in exemplary aspects, each longitudinal support assembly 40 can comprise a single tube, and the first and second end portions 46, 48 of the longitudinal support assembly can comprise opposed ends of the tube.

In an additional aspect, the shaft 42 of each longitudinal support assembly 40 of the at least one longitudinal support assembly can have a central axis 50. In this aspect, and with reference to FIGS. 3 and 6A, the first and second end portions 46, 48 of each longitudinal support assembly 40 can be selectively moveable relative to the central axes 24a, 24b of the shafts 22a, 22b of the first and second transverse support assemblies 20a, 20b to position the central axis 50 of the longitudinal support assembly at a selected angle 52 relative to the longitudinal axis 102 of the table 100. In exemplary aspects, the selected angle 52 can range from 0° to about 45°.

In another aspect, and with reference to FIGS. 3 and 6A, each longitudinal support assembly 40 can have a length that is adjustable to permit independent axial movement of the first and second end portions 46, 48 of the longitudinal support assembly relative to the respective central axes 24a, 24b of the shafts 22a, 22b of the first and second transverse support assemblies 20a, 20b.

Optionally, in exemplary aspects, and with reference to FIGS. 2-3 and 5A-6B, the first and second end portions 46, 48 of each longitudinal support assembly 40 can be respectively pivotally coupled to the shafts 22a, 22b of the first and second transverse support assemblies 20a, 20b. Optionally, in these aspects, and as shown in FIGS. 6A-6B, the shaft 42 of each longitudinal support assembly 40 of the at least one longitudinal support assembly can define a central conduit 54, and at least one of the first end portion 46 and the second end portion 48 of the longitudinal support assembly can comprise an elongate element 56 that is at least partially received within the central conduit of the shaft and slidably coupled to the shaft to permit adjustment of the length of the longitudinal support assembly. Thus, it is contemplated that at least one of the first end portion 46 and the second end portion 48 can be a discrete component of the longitudinal support assembly 40, with at least one of the first end portion and the second end portion positioned within the shaft 42 in a tube-within-a-tube configuration. Optionally, both the first end portion 46 and the second end portion 48 of each longitudinal support assembly 40 of the at least one longitudinal support assembly can comprise a respective elongate element 56 that is at least partially received within the central conduit 54 of the shaft 42 and slidably coupled to the shaft to permit adjustment of the length of the longitudinal support assembly.

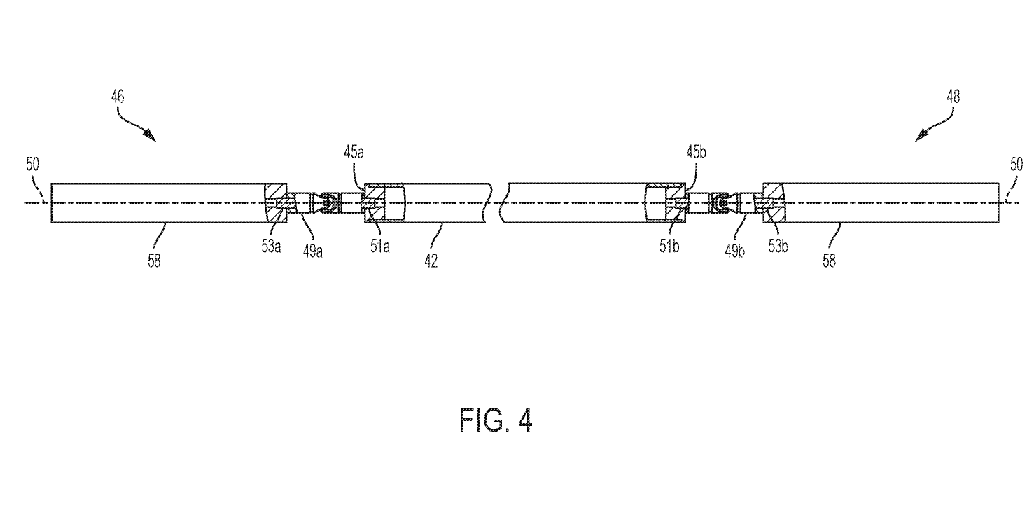

Alternatively, in a further optional aspect, and as shown in FIGS. 3-5B, both the first and second end portions 46, 48 of each longitudinal support assembly 40 can comprise a respective elongate element 58 that is pivotally coupled to the shaft 42 of the longitudinal support assembly. As shown, in this configuration, each longitudinal support assembly 40 can effectively comprise three tubes that are pivotally connected to one another. Optionally, in exemplary aspects, and as shown in FIG. 4, each longitudinal support assembly 40 can further comprise a first joint mount 45a secured to a first end of the shaft 42 and a second joint mount 45b secured to an opposed second end of the shaft. The first joint mount 45a can engage a first stud 51a, and the second joint mount 45b can engage a second stud 51b. Each longitudinal support assembly 40 can further comprise U-joints 49a, 49b that are secured to inner ends (the ends closest to the shaft 42) of the elongate elements 58 and configured for operative pivotal coupling to a portion of the joint mounts 45a, 45b. The U-joints 49a, 49b can be secured to the inner ends of the elongate elements 58 using third and fourth studs 53a, 53b. The exemplary configuration depicted in FIG. 4 is merely exemplary, and it is contemplated that any conventional pivotal connection can be used to connect the shaft 42 to the elongate elements 58. Exemplary pivotal connections include, for example and without limitation, ball joints, universal joints, knuckle joints, and combinations thereof.

In another aspect, and with reference to FIGS. 2-3 and 7B, it is contemplated that the shafts 22a, 22b of both the first and second transverse support assemblies 20a, 20b can be respectively adjustably coupled to the first and second end portions 46, 48 of each longitudinal support assembly to permit adjustment of the position of the first and second transverse support assemblies relative to the longitudinal axis 102 of the table 100. Additionally or alternatively, it is contemplated that the first and second end portions 46, 48 of each longitudinal support assembly can be respectively adjustably coupled to the shafts 22a, 22b of both the first and second transverse support assemblies 20a, 20b to permit adjustment of the position of the shaft 42 relative to the longitudinal axis 102 of the table 100.

In exemplary aspects, and as shown in FIG. 3, the frame 10 can further comprise at least first and second pairs 61a, 61b of opposed support posts 60. In these aspects, each support post 60 can have a first end 62 and an opposed second end 64. It is contemplated that the first end 62 of each support post 60 can be operatively connectable to the table 100. In exemplary aspects, and as shown in FIG. 3, the first end 62 of each support post 60 can comprise an outer portion (optionally, an outer portion having a reduced diameter) that is configured for receipt within a receptacle defined by the proximal or distal side of the table 100. Alternatively, the first end 62 of each support post 60 can comprise a conventional connector or fastener as is known in the art for adjustably coupling the support post to the proximal or distal side of the table 100. It is further contemplated that each support post 60 can extend upwardly from the first end 62 of the support post such that the second end 64 of the support post is spaced from the first end of the support post relative to the vertical axis 114. As shown in FIG. 3, each pair 61a, 61b of opposed support posts 60 can have a proximal support post 66 and a distal support post 68 that are spaced apart relative to the transverse axis 104 of the table 100. In exemplary aspects, the proximal support post 66 can be operatively connectable to the proximal side 110 of the table 100, and the distal support post 68 can be operatively connectable to the distal side 112 of the table. In further exemplary aspects, the first pair 61a of opposed support posts 60 can be operatively coupled to and configured to support the first transverse support assembly 20a, and the second pair 61b of opposed support posts can be operatively coupled to and configured to support the second transverse support assembly 20b. Optionally, in still further exemplary aspects, at least one support post 60 of the frame 10 can comprise at least one connector (not shown) securely coupled to the support post and configured to engage and support a surgical tool.

In another aspect, and with reference to FIG. 3, the shafts 22a, 22b of both the first and second transverse support assemblies 20a, 20b can be respectively adjustably coupled to the first and second pairs 61a, 61b of opposed support posts 60 to permit adjustment of the position of the first and second transverse support assemblies relative to the vertical axis 114. Thus, it is contemplated that the first transverse support assembly 20a can be positioned at the same height or a different height (relative to the vertical axis) than the second transverse support assembly 20b. Optionally, the shafts 22a, 22b of both the first and second transverse support assemblies 20a, 20b can have a first end 26a, 26b and an opposed second end 28a, 28b. The first ends 26a, 26b of the shafts 22a, 22b of the first and second transverse support assemblies 20a, 20b can be respectively adjustably coupled to the proximal support posts 66 of the first and second pairs 61a, 61b of opposed support posts 60, and the second ends 28a, 28b of the shafts of the first and second transverse support assemblies can be respectively adjustably coupled to the distal support posts 68 of the first and second pairs of opposed support posts. It is contemplated that the respective positions of the first ends 26a, 26b of the first and second transverse support assemblies 20a, 20b relative to the proximal support posts 66 of the first and second pairs 61a, 61b of opposed support posts 60 can be adjustable independently of the respective positions of the second ends 28a, 28b of the first and second transverse assemblies relative to the distal support posts 68 of the first and second pairs of opposed support posts. In exemplary aspects, the first and second ends 26a, 26b, 28a, 28b of the shafts 22a, 22b of the first and second transverse support assemblies 20a, 20b can be selectively moveable relative to the vertical axis 114 to position the central axis 24a, 24b of the shaft of each transverse support assembly at a selected angle 30 relative to the transverse axis 104. Optionally, in these aspects, the selected angle 30 can range from 0° to about 45° relative to the transverse axis 104 (which corresponds to an angle of 45° to about 90° relative to the vertical axis).

In exemplary aspects, and with reference to FIGS. 3 and 9, the frame 10 can further comprise a plurality of fixation assemblies 80. In these aspects, the plurality of fixation assemblies 80 can operatively couple the at least one longitudinal support assembly 40 to the first and second transverse support assemblies 20a, 20b. Optionally, in one aspect, the plurality of fixation assemblies 80 can comprise a first receptacle 82 and a second receptacle 84 that is oriented in a direction that is substantially perpendicular to an orientation direction of the first receptacle. For example, in this aspect, it is contemplated that the first receptacle 82 can be configured to receive an end portion 46, 48 of a respective longitudinal support assembly 40 and the second receptacle 84 can be configured to receive the shaft 22a, 22b of a respective transverse support assembly 20a, 20b. In use, it is contemplated that end portions 46, 48 of a longitudinal support assembly can be configured for sliding movement within a first receptacle 82 relative to the longitudinal axis of the table to permit selective adjustment of the operative length of the longitudinal support assembly such that the shaft of the longitudinal support assembly can be angled as desired relative to the longitudinal axis of the table. In exemplary aspects, when the frame 10 comprises a plurality of support posts 60 as disclosed herein, the frame can comprise a first plurality of fixation assemblies 80 and a second plurality of fixation assemblies. In these aspects, the first plurality of fixation assemblies 80 can operatively couple the at least one longitudinal support assembly 40 to the first and second transverse support assemblies 20a, 20b. In these aspects, each fixation assembly 80 of the first plurality of fixation assemblies can comprise a first receptacle 82 configured to receive an end portion 46, 48 of a respective longitudinal support assembly 40 and a second receptacle 84 configured to receive the shaft 22a, 22b of a respective transverse support assembly 20a, 20b. In additional aspects, the second plurality of fixation assemblies 80 can operatively couple the first and second transverse support assemblies 20a, 20b to the first and second pairs 61a, 61b of opposed support posts 60. In these aspects, each fixation assembly 80 of the second plurality of fixation assemblies can comprise a first receptacle 82 configured to receive the shaft 22a, 22b of a respective transverse support assembly 20a, 20b and a second receptacle 84 configured to receive a respective support post 60. In exemplary aspects, each receptacle 82, 84 can comprise a first portion and a second portion that cooperate to define a central space for receiving the shaft of a support assembly or a support post. The first and second portions can have first ends that are joined to one another and opposed second ends that are spaced apart and coupled together by a fastener 86. Each fixation assembly 80 can have a cam handle 83, 85 that is operatively coupled to the fastener 86 such that selective movement of the cam handle is configured to effect inward or outward radial movement of the second ends of the first and second portions of the receptacle, thereby permitting releasable secure engagement of a shaft of a support assembly or support post (and adjustment of the axial position of the fixation assembly 80 relative to the central axis of the support assembly or the support post).

Although the fixation assembly 80 is described as comprising a cam handle, it is contemplated that any conventional clamping mechanism can be used. Exemplary clamping mechanisms include, for example and without limitation, threaded handles, compression clamping mechanisms, ratcheting mechanisms, split collar clamps, spring clamps, screw clamps, toggle clamps, open tube clamps, and combinations thereof. In use, it is contemplated that each fixation assembly 80 can be selectively removable and attachable to a shaft of a support assembly or support post, while leaving the shaft of the support assembly or the support post in place. It is further contemplated that each fixation assembly 80 can be selectively removed or attached to a shaft of a support assembly or support post without the use of a securing tool.

Optionally, as shown in FIGS. 6A-6B, when at least one of the first end portion 46 and the second end portion 48 of the longitudinal support assembly comprises an elongate element 56 that is at least partially received within the central conduit 54 of the shaft 42 of the longitudinal support assembly and is slidably coupled to the shaft to permit adjustment of the length of the longitudinal support assembly, each fixation assembly 80 can comprise a pivotal connection and a single receptacle that is operatively secured to the pivotal connection. The pivotal connection can be operatively coupled to the elongate element 56, and the single receptacle can be configured to receive a shaft 22a, 22b of a transverse support assembly 20a, 20b. In exemplary aspects, it is contemplated that the pivotal connection can comprise a ball joint, a universal joint, a knuckle joint, and combinations thereof. However, it is contemplated that any conventional pivotal connection can be used.

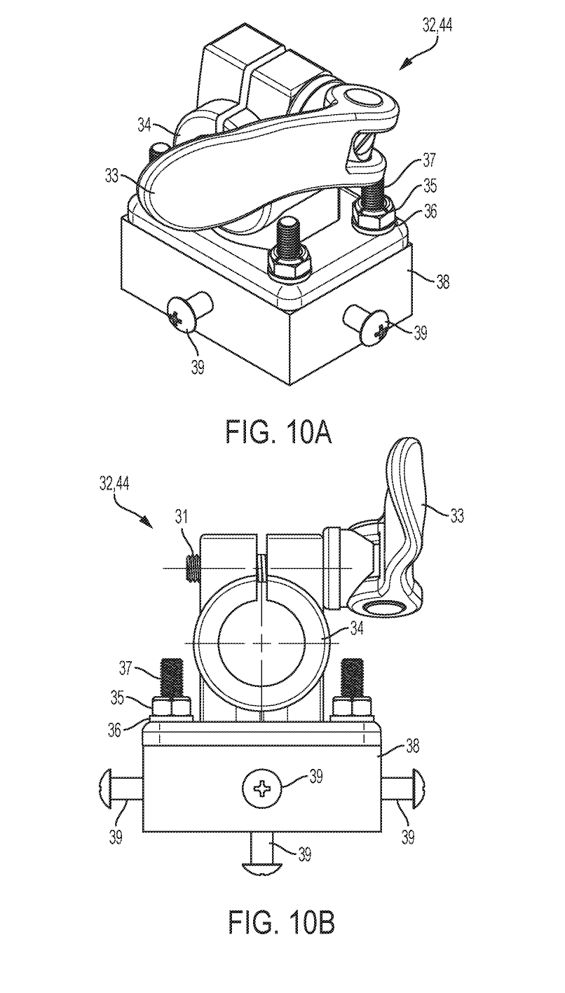

An exemplary configuration of the connectors 32, 44 and the connectors of the support posts (not shown) is depicted in FIGS. 10A-10B. As shown, each connector 32, 44 can optionally be a lever action cam connector. Optionally, in exemplary aspects, the connector 32, 44 can comprise a receptacle 34 that is configured to receive the shaft of a respective transverse support assembly, longitudinal support assembly, or support post. The receptacle 34 can comprise a first portion and a second portion that cooperate to define a central space for receiving the shaft of the support assembly or support post. The first and second portions can have first ends that are secured to a clamp plate 38 and opposed second ends that are spaced apart and coupled together by a fastener 31. Each connector 32, 44 can have a cam handle 33 that is operatively coupled to the fastener 31 such that selective movement of the cam handle is configured to effect inward or outward radial movement of the second ends of the first and second portions of the receptacle, thereby permitting releasable secure engagement of a shaft of a support assembly or support post (and adjustment of the axial position of the connector 32, 44 relative to the central axis of the support assembly or the vertical axis, in the case of a support post). The first ends of the first and second portions of the receptacle 34 can be secured to the clamp plate 38 using a coupling assembly comprising a nut 35, a washer 36, and a screw 37 as depicted in FIGS. 10A-10B. The clamp plate 38 can comprise at least one screw 39 that is configured for operative engagement with a surgical tool in the manner known in the art. Although each connector 32, 44 is described as comprising a cam handle, it is contemplated that any conventional clamping mechanism can be used. Exemplary clamping mechanisms include, for example and without limitation, threaded handles, compression clamping mechanisms, ratcheting mechanisms, split collar clamps, spring clamps, screw clamps, toggle clamps, open tube clamps, and combinations thereof. In use, it is contemplated that each connector 32, 44 can be selectively removable and attachable to a shaft of a support assembly or a support post, while leaving the shaft of the support assembly or support post in place. It is further contemplated that each connector 32, 44 can be selectively removed or attached to a shaft of a support assembly or support post without the use of a securing tool.

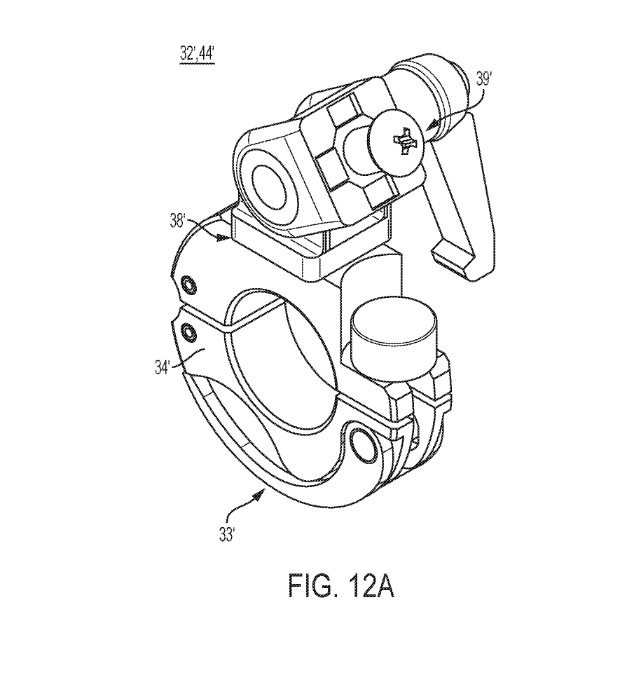

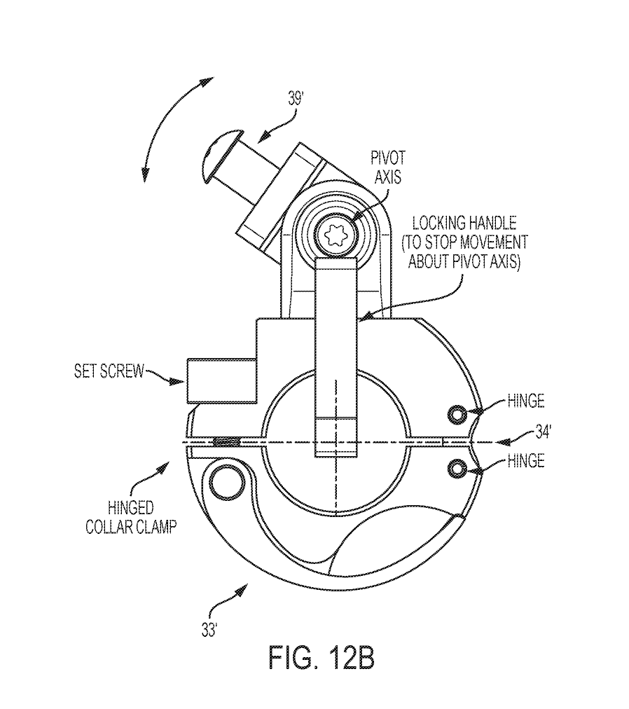

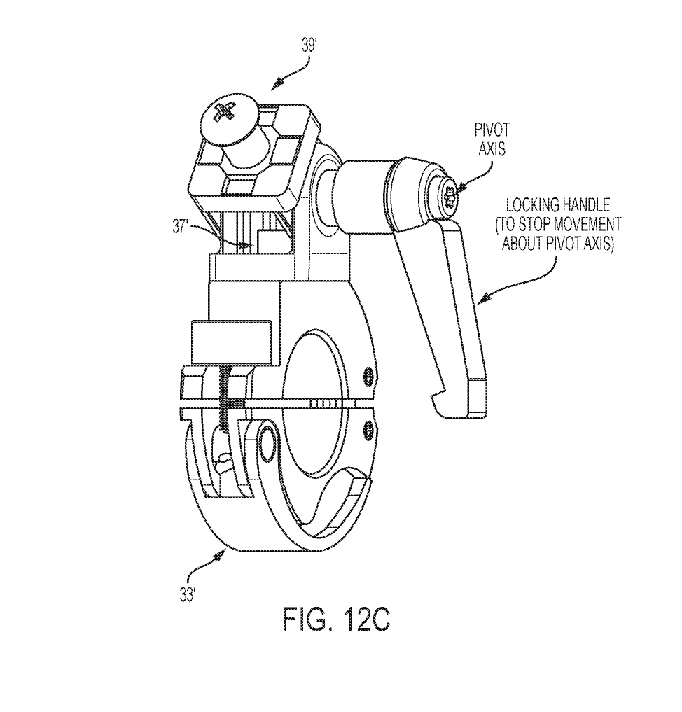

An alternative exemplary configuration of the connectors 32′, 44′ and the connectors of the support posts (not shown) is depicted in FIGS. 12A-12C. As shown, the connector is composed of a modified hinged shaft collar clamp with a locking pivot for the connector to interface with a surgical tool. It is contemplated that the connectors 32′, 44′, can have a smaller volume to minimize interference with the positioning of a surgical tool. The connector 32′, 44′ can comprise a receptacle 34′ that is configured to receive the shaft of a respective transverse support assembly, longitudinal support assembly, or support post. The receptacle 34′ can comprise a first portion and a second portion that cooperate to define a central space for receiving the shaft of the support assembly or support post. The first and second portions can have first ends that are secured to a clamp plate 38′ and opposed second ends that are spaced apart and coupled together by a hinge and set screw. Each connector 32′, 44′ can have a cam handle 33′ that is operatively coupled to the set screw and hinge such that selective movement of the cam handle is configured to effect inward or outward radial movement of the second ends of the first and second portions of the receptacle, thereby permitting releasable secure engagement of a shaft of a support assembly or support post (and adjustment of the axial position of the connector 32′, 44′ relative to the central axis of the support assembly or the vertical axis, in the case of a support post). The hinged connector can be attached/detached by engaging/disengaging the cam handle 33′ and positioning the connector around the shaft of a transverse support assembly, longitudinal support assembly or support post, preferentially without disassembly of the frame 10. An attachment screw 39′ may be pivoted about an axis parallel to receptacle 34′ and locked into place via a locking handle. Although the connector 32′, 33′ is described as comprising a cam handle, it is contemplated that any conventional clamping mechanism can be used. Exemplary clamping mechanisms include, for example and without limitation, threaded handles, compression clamping mechanisms, ratcheting mechanisms, split collar clamps, spring clamps, screw clamps, toggle clamps, open tube clamps, and combinations thereof. Although described above as extending between and being operatively coupled to respective transverse support assemblies, it is contemplated that each longitudinal support assembly can alternatively extend between and be operatively coupled to respective support posts that are spaced apart relative to the longitudinal axis of the table. In exemplary aspects, it is contemplated that at least one longitudinal support assembly can extend between and be operatively coupled to first and second transverse support assemblies and that at least one longitudinal support assembly can extend between and be operatively coupled to first and second proximal support posts (or first and second distal support posts) that are spaced apart relative to the longitudinal axis of the table.

Optionally, in exemplary aspects, it is contemplated that the at least first and second transverse support assemblies can comprise first, second, and third transverse support assemblies spaced apart relative to the longitudinal axis of the table, with the second transverse support assembly positioned between the first and third transverse support assemblies relative to the longitudinal axis of the table. Optionally, in these aspects, a first longitudinal support assembly can extend between and be operatively coupled to the first and second transverse support assemblies, while a second longitudinal support assembly can extend between and be operatively coupled to the second and third transverse support assemblies.

### Frames Having a Single Transverse Support Assembly

Optionally, rather than having first and second transverse support assemblies that are spaced apart relative to the longitudinal axis of the table, it is contemplated that at least one of the first and second transverse support assemblies can be eliminated and replaced with a support post that is advantageously positioned at the proximal side of the table, at the distal side of the table, or between the proximal and distal sides of the table. In these configurations, it is contemplated that the other components of the frame, including the transverse and longitudinal support assemblies, the connectors, and support posts, can be substantially the same as those previously described herein.

In exemplary aspects, as shown in FIG. 11, the frame 10 can comprise a first transverse support assembly 20 that is configured to extend from the proximal side 110 of the table to the distal side 112 of the table. In these aspects, the first transverse support assembly 22 can have a shaft 22 with a central axis 24. Optionally, the frame can comprise at least one connector 32 securely coupled to the shaft of the first transverse support assembly 22 and configured to engage and support a surgical tool. Alternatively, no connectors 32 are coupled to the shaft of the first transverse support assembly 22.

In another aspect, the frame 10 can comprise a plurality of support posts 60. In this aspect, the plurality of support posts 66 can comprise at least three support posts. Each support post 60 can extend upwardly from the first end of the support post such that the second end of the support post is spaced from the first end of the support post relative to the vertical axis. In further aspects, the at least three support posts can comprise a pair of opposed support posts having a proximal support post and a distal support post that are spaced apart relative to the transverse axis 104 of the table 100. In these aspects, the first end 62 of the proximal support post can be operatively connectable to the proximal side 110 of the table and the first end 62 of the distal support post can be operatively connectable to the distal side 112 of the table. The pair of opposed support posts can be operatively coupled to and configured to support the first transverse support assembly 20. In additional aspects, the at least three support posts can further comprise at least a third support post spaced from the pair of opposed support posts relative to the longitudinal axis 102 of the table 100. In these aspects, the first end of the third support post can be secured to the table at a selected position. It is contemplated that the selected position can be selected from the group consisting of: a position at the proximal side of the table; a position at the distal side of the table; and a position between the proximal and distal sides of the table relative to the transverse axis of the table. Optionally, in exemplary aspects, it is contemplated that the first end 62 of the third support post can be secured to the table 100 at a substantially midline position. In these aspects, it is contemplated that the legs or feet of a subject can be positioned on opposite sides of the third support post during a surgical procedure.

In yet another aspect, and with reference to FIG. 11, the frame can comprise at least one longitudinal support assembly 40 extending between and operatively coupled to the first transverse support assembly and the third support post. In this aspect, the at least one longitudinal support assembly 40 can comprise a shaft 42 and at least one connector 44 (as further disclosed herein) that is securely coupled to the shaft and configured to engage and support a surgical tool. In exemplary aspects, the at least one connector coupled to the shaft 42 can comprise a plurality of connectors.

In operation, the frame can be connectable to the surgical table such that the first support assembly and the at least one longitudinal support assembly are spaced from the surgical table in an upward direction relative to the vertical axis.

In a further aspect, and with reference to FIG. 11, each longitudinal support assembly 40 of the at least one longitudinal support assembly can have a first end portion 46 operatively coupled to the shaft 22 of the first transverse support assembly 20 and a second end portion 48 operatively coupled to the third support post. In this aspect, the first end portion of the longitudinal support assembly can be selectively axially moveable relative to the central axis of the shaft of the first transverse support assembly, and the second end portion of the longitudinal support assembly can be pivotally coupled to the third support post.

Optionally, in exemplary aspects, each longitudinal support assembly 40 can comprise a single tube, and the first and second end portions 46, 48 of the longitudinal support assembly can comprise opposed ends of the tube.

In an additional aspect, the shaft 42 of each longitudinal support assembly 40 of the at least one longitudinal support assembly can have a central axis 50. In this aspect, the first end portion 46 of each longitudinal support assembly 40 can be selectively moveable relative to the central axis 24 of the shaft 22 of the first transverse support assembly 20, and the second end portion 48 of each longitudinal support assembly can be pivoted about the third support post to position the central axis 50 of the longitudinal support assembly 40 at a selected angle relative to the longitudinal axis 102 of the table 100. In exemplary aspects, the selected angle can range from 0° to about 45°.

In another aspect, each longitudinal support assembly can have a length that is adjustable to permit selective axial movement of the first end portion of the longitudinal support assembly relative to the central axis of the shaft of the first transverse support assembly while the second end portion of the longitudinal support assembly is pivotally coupled to the third support post.

Optionally, in exemplary aspects, the first end portion 46 of each longitudinal support assembly 40 can be pivotally coupled to the shaft 22 of the first transverse support assembly 20, and the second end portion 48 of each longitudinal support assembly 40 can be pivotally coupled to the third support post. Optionally, in these aspects, the shaft of each longitudinal support assembly of the at least one longitudinal support assembly can define a central conduit, and at least one of the first end portion and the second end portion of the longitudinal support assembly can comprise an elongate element that is at least partially received within the central conduit of the shaft and slidably coupled to the shaft to permit adjustment of the length of the longitudinal support assembly. Thus, it is contemplated that at least one of the first end portion and the second end portion can be a discrete component of the longitudinal support assembly, with at least one of the first end portion and the second end portion positioned within the shaft in a tube-within-a-tube configuration. Optionally, both the first end portion and the second end portion of each longitudinal support assembly of the at least one longitudinal support assembly can comprise a respective elongate element that is at least partially received within the central conduit of the shaft and slidably coupled to the shaft to permit adjustment of the length of the longitudinal support assembly.

Alternatively, in a further optional aspect, both the first and second end portions of each longitudinal support assembly can comprise a respective elongate element that is pivotally coupled to the shaft of the longitudinal support assembly in the manner disclosed above with respect to frame 10. In this configuration, each longitudinal support assembly can effectively comprise three tubes that are pivotally connected to one another.

In another aspect, it is contemplated that the shaft of the first transverse support assembly can be adjustably coupled to the first and second end portions of each longitudinal support assembly to permit adjustment of the position of the first and second transverse support assemblies relative to the longitudinal axis of the table.

In exemplary aspects, the first end of each of the proximal and distal support posts can comprise an outer portion (optionally, a reduced diameter portion) that is configured for receipt within a receptacle defined by the proximal or distal side of the table. Alternatively, the first end of each of the proximal and distal support posts can comprise a conventional connector for adjustably coupling the support post to the proximal or distal side of the table. Optionally, in still further exemplary aspects, at least one support post of the frame can comprise at least one connector securely coupled to the support post and configured to engage and support a surgical tool.

In another aspect, the shaft of the first transverse support assembly can be adjustably coupled to the pair of opposed support posts to permit adjustment of the position of the first transverse support assembly relative to the vertical axis. Optionally, the shaft of the first transverse support assembly can have a first end and an opposed second end. The first end of the shaft of the first transverse support assembly can be adjustably coupled to the proximal support post of the pair of opposed support posts, and the second end of the shaft of the first transverse support assembly can be respectively adjustably coupled to the distal support post of the pair of opposed support posts. It is contemplated that the position of the first end of the first transverse support assembly relative to the proximal support post of the pair of opposed support posts can be adjustable independently of the position of the second end of the first transverse assembly relative to the distal support post of the pair of opposed support posts. In exemplary aspects, the first and second ends of the shaft of the first transverse support assembly can be selectively moveable relative to the vertical axis to position the central axis of the shaft of the first transverse support assembly at a selected angle relative to the transverse axis. Optionally, in these aspects, the selected angle can range from 0° to about 45° relative to the transverse axis (which corresponds to an angle of 45° to about 90° relative to the vertical axis).

In exemplary aspects, the frame can further comprise a plurality of fixation assemblies as disclosed above with respect to frame 10. In these aspects, the plurality of fixation assemblies can operatively couple the at least one longitudinal support assembly to the first transverse support assembly and the third support post. In exemplary aspects, when the frame comprises a plurality of support posts as disclosed herein, the frame can comprise a first plurality of fixation assemblies and a second plurality of fixation assemblies. In these aspects, the first plurality of fixation assemblies can operatively couple the at least one longitudinal support assembly to the first transverse support assembly and the third support post. In additional aspects, the second plurality of fixation assemblies can operatively couple the first transverse support assembly to the pair of opposed support posts.

Optionally, when at least one of the first end portion and the second end portion of the longitudinal support assembly comprises an elongate element that is at least partially received within the central conduit of the shaft of the longitudinal support assembly and is slidably coupled to the shaft to permit adjustment of the length of the longitudinal support assembly, the plurality of fixation assemblies can comprise at least one fixation assembly having a pivotal connection and a single receptacle that is operatively secured to the pivotal connection. The pivotal connection can be operatively coupled to the elongate element, and the single receptacle can be configured to receive a portion of the third support post or the shaft of the first transverse support assembly. In exemplary aspects, it is contemplated that the pivotal connection can comprise a ball joint, a universal joint, a knuckle joint, and combinations thereof. However, it is contemplated that any conventional pivotal connection can be used.

### Exemplary Frame Materials

Optionally, in some exemplary aspects, at least portions of the disclosed frame (e.g., frame 10) can comprise radiopaque materials, such as, for example and without limitation, aluminum, titanium, stainless steel, or alloys containing nickel, cobalt, chromium, molybdenum, and/or vanadium, and the like.

Optionally, in exemplary aspects, portions of the disclosed frame (e.g., frame 10) can comprise radiolucent materials or, more preferably, radiotransparent materials. As used herein, “radiopaque” refers to materials through which a substantial amount of electromagnetic radiation is attenuated, and through which little to no radiation passes. Thus, objects in an imaging pathway may not be visible through a “radiopaque” material by the electromagnetic radiation. As used herein, “radiolucent” refers to materials through which some amount of electromagnetic radiation is attenuated, and through which some radiation passes. Thus, objects in an imaging pathway may be visible through a “radiolucent” material by the electromagnetic radiation, but some details of the objects may not be discernible. As used herein, “radiotransparent” refers to materials through which substantially no electromagnetic radiation is attenuated, and through which substantially all of the radiation passes. Thus, objects in an imaging pathway may be clearly visible through a “radiotransparent” material by the electromagnetic radiation, and the details of the objects may be clearly visible and/or discernible.

In some aspects, it is contemplated that the disclosed longitudinal support assemblies (e.g., longitudinal support assembly 40) can be radiolucent or, more preferably, radiotransparent. Additionally, it is contemplated that the connectors 44, 44′ coupled to the longitudinal support assemblies 40 can be radiolucent or, more preferably, radiotransparent. In further aspects, at least portions of the transverse support assemblies (e.g., first and second transverse support assemblies 20a, 20b) and support posts (e.g., support posts 60) can be radiolucent or, more preferably, radiotransparent. In still further aspects, it is contemplated that at least portions of the disclosed connectors 32, 32′ and fixation assemblies (e.g., fixation assemblies 80) can be radiolucent or, more preferably, radiotransparent.

In use, it is contemplated that a frame having radiotransparent or radiolucent portions can allow surgeons to produce X-ray images without the need for disassembling the frame, as is required during medical procedures performed with conventional frames. For example, by using radiotransparent or radiolucent longitudinal support assemblies 40 and connectors 44, a clinician can produce X-ray images while leaving the longitudinal support assemblies and connectors in a desired position over or within a surgical field (during the performance of a surgical procedure on the patient). Exemplary radiolucent materials that can be used to form elements of the frame include radiolucent composite materials, radiolucent polymer materials (polyesters, epoxies, polyaramids, poly ether ether ketones, polycarbonates, polyoxymethylenes, polyethylenes, polypropylenes, polybutalene terephthalates, polyimides, etc.) with or without reinforcing materials, radiolucent ceramic materials, carbon fibers, and the like. It is contemplated that radiolucency and/or radiotransparency can depend on the density (related to the atomic number) of the material and the path length (material thickness). Thus, it is contemplated that a radiotransparent material can become radiolucent if sufficient material is added to it.

### Surgical Retraction Systems

In exemplary aspects, the disclosed frame 10 can be provided as part of a surgical retraction system. In these aspects, the surgical retraction system can comprise the frame 10 and at least one surgical tool that is secured to and supported by a connector of the frame. In exemplary aspects, it is contemplated that the at least one surgical tool can comprise a surgical retractor as is known in the art. In an additional aspect, it is contemplated that the surgical retraction system can further comprise a surgical table as described herein.

### Methods

In further exemplary aspects, the disclosed frame 10 can be employed in a method of using one or more surgical tools. In these aspects, the method can comprise securing the frame to a surgical table. In another aspect, the method can comprise securing at least one surgical tool to a connector of the frame. In another aspect, the method can further comprise selectively activating the at least one surgical tool. In this aspect, it is contemplated that the at least one surgical tool can comprise a surgical retractor as is known in the art.

In exemplary applications, the disclosed frame can be used to perform a surgical retraction procedure, such as, for example and without limitation, an acetabulum retraction, a pelvic ring retraction, a femur retraction, a tibial plateau procedure, a spinal procedure, and the like. In exemplary aspects, the frame can be used to hold various retractors in an anterior intrapelvic approach. Non-limiting advantages of using the disclosed frame include needing fewer surgical assistants during the procedure to hold retractors and increased stability/decreased movement of the retractors after placement.

In use, the disclosed frame can be configured to be cleaned using conventional means, such as, for example and without limitation, cleaning detergents and/or solvents, manual and/or automatic cleaning tools, ultrasonic washing, and the like. The disclosed frame can also be configured to be sterilized using conventional means, such as, for example and without limitation, steam heat, ethylene oxide, dry heat, microwave radiation, plasma, ozone, chemical, ionizing radiation, and the like. In exemplary aspects, the frame 10 can be configured to be disassembled to fit within conventional sterilization equipment.

### Exemplary, Non-Limiting Dimensions and Characteristics

Optionally, in exemplary aspects, it is contemplated that the disclosed frame can be configured to withstand at least 75 pounds of force on each support assembly 20a, 20b, 40 with a deflection of no more than 0.125 inches and, more preferably, a deflection of no more than 0.075 inches. In other exemplary aspects, it is contemplated that the disclosed frame can be configured to withstand at least 50 pounds of force on each support assembly 20a, 20b, 40 with a deflection of no more than 0.05 inches

Optionally, in exemplary aspects, the longitudinal support assembly 40 can have an axial length of at least 3 inches relative to the longitudinal axis of the table. Optionally, it is contemplated that the axial length of the longitudinal support assembly 40 can be at least 4 inches relative to the longitudinal axis of the table. In other optional aspects, it is contemplated that the axial length of the longitudinal assembly 40 can range from about 3 inches to about 40 inches.

Optionally, in additional exemplary aspects, the transverse support assemblies 20a, 20b can have an axial length of at least 1 inch relative to the transverse axis of the table. Optionally, the axial length of the transverse assemblies 20a, 20b can be at least 2 inches. In other optional aspects, it is contemplated that the axial length of the transverse support assemblies 20a, 20b can range from about 1 inch to about 50 inches.

Optionally, in further exemplary aspects, the support posts 60 can have an axial length of at least one inch relative to the vertical axis. Optionally, the axial length of the support posts 60 can be at least 2 inches. In other optional aspects, it is contemplated that the axial length of the support posts can range from about 1 inch to about 40 inches.

### Exemplary Aspects

In view of the described frames, systems, and methods and variations thereof, herein below are described certain more particularly described aspects of the invention. These particularly recited aspects should not however be interpreted to have any limiting effect on any different claims containing different or more general teachings described herein, or that the “particular” aspects are somehow limited in some way other than the inherent meanings of the language literally used therein.

Aspect 1: A frame for supporting one or more surgical tools during a surgical procedure, the frame being connectable to a surgical table and comprising: first and second transverse support assemblies, each transverse support assembly being configured to extend from a proximal side of a surgical table to an opposed distal side of the surgical table and having a shaft with a central axis, wherein the second transverse support assembly is spaced from the first transverse support assembly relative to a longitudinal axis of the surgical table, wherein the proximal and distal sides of the surgical table extend between opposed first and second ends of the surgical table, and wherein the proximal side of the surgical table is spaced apart from the distal side of the surgical table relative to a transverse axis that is substantially perpendicular to the longitudinal axis of the surgical table; and at least one longitudinal support assembly extending between and operatively coupled to the first and second transverse support assemblies, wherein the at least one longitudinal support assembly comprises a shaft and at least one connector securely coupled to the shaft and configured to engage and support a surgical tool, wherein the frame is connectable to the surgical table such that the first and second transverse support assemblies and the at least one longitudinal support assembly are spaced from the surgical table in an upward direction relative to a vertical axis that is substantially perpendicular to both the longitudinal axis of the surgical table and a transverse axis of the surgical table.

Aspect 2: The frame of aspect 1, wherein the at least one longitudinal support assembly is radiolucent.

Aspect 3: The frame of any one of the preceding aspects, wherein each longitudinal support assembly of the at least one longitudinal support assembly has a first end portion operatively coupled to the shaft of the first transverse support assembly and a second end portion operatively coupled to the shaft of the second transverse support assembly, wherein the first end portion of the longitudinal support assembly is selectively axially moveable relative to the central axis of the shaft of the first transverse support assembly, and wherein the second end portion of the longitudinal support assembly is selectively axially moveable relative to the central axis of the shaft of the second transverse support assembly.

Aspect 4: The frame of aspect 3, wherein the shaft of each longitudinal support assembly of the at least one longitudinal support assembly has a central axis, wherein the first and second end portions of each longitudinal support assembly are selectively moveable relative to the central axes of the shafts of the first and second transverse support assemblies to position the central axis of the longitudinal support assembly at a selected angle relative to the longitudinal axis of the table, wherein the selected angle ranges from 0° to about 45°.

Aspect 5: The frame of aspect 3, wherein each longitudinal support assembly has a length that is adjustable to permit independent axial movement of the first and second end portions of the longitudinal support assembly relative to the respective central axes of the shafts of the first and second transverse support assemblies.

Aspect 6: The frame of aspect 5, wherein the first and second end portions of each longitudinal support assembly are respectively pivotally coupled to the shafts of the first and second transverse support assemblies.

Aspect 7: The frame of aspect 6, wherein the shaft of each longitudinal support assembly of the at least one longitudinal support assembly defines a central conduit, and wherein at least one of the first end portion and the second end portion of the longitudinal support element comprises an elongate element that is at least partially received within the central conduit of the shaft and slidably coupled to the shaft to permit adjustment of the length of the longitudinal support assembly.

Aspect 8: The frame of aspect 7, wherein both the first end portion and the second end portion of each longitudinal support assembly of the at least one longitudinal support assembly comprise a respective elongate element that is at least partially received within the central conduit of the shaft and slidably coupled to the shaft to permit adjustment of the length of the longitudinal support assembly.

Aspect 9: The frame of aspect 3, wherein both the first and second end portions of each longitudinal support assembly comprise a respective elongate element that is pivotally coupled to the shaft of the longitudinal support assembly.

Aspect 10: The frame of aspect 3, wherein the shafts of both the first and second transverse support assemblies are respectively adjustably coupled to the first and second end portions of each longitudinal support assembly to permit adjustment of the position of the first and second transverse support assemblies relative to the longitudinal axis of the table.

Aspect 11: The frame of any one of the preceding aspects, wherein the frame further comprises: at least first and second pairs of opposed support posts, each support post having a first end and an opposed second end, wherein the first end of each support post is operatively connectable to the table, wherein each support post extends upwardly from the first end of the support post such that the second end of the support post is spaced from the first end of the support post relative to the vertical axis, wherein each pair of opposed support posts has a proximal support post and a distal support post that are spaced apart relative to the transverse axis of the table, wherein the proximal support post is operatively connectable to the proximal side of the table and the distal support post is operatively connectable to the distal side of the table, wherein the first pair of opposed support posts is operatively coupled to and configured to support the first transverse support assembly, and wherein the second pair of opposed support posts is operatively coupled to and configured to support the second transverse support assembly.

Aspect 12: The frame of aspect 11, wherein the shafts of both the first and second transverse support assemblies are respectively adjustably coupled to the first and second pairs of opposed support posts to permit adjustment of the position of the first and second transverse support assemblies relative to the vertical axis.

Aspect 13: The frame of aspect 12, wherein the shafts of both the first and second transverse support assemblies have opposed first and second ends, wherein the first ends of the shafts of the first and second transverse support assemblies are respectively adjustably coupled to the proximal support posts of the first and second pairs of opposed support posts, wherein the second ends of the shafts of the first and second transverse support assemblies are respectively adjustably coupled to the distal support posts of the first and second pairs of opposed support posts, and wherein the respective positions of the first ends of the first and second transverse support assemblies relative to the proximal support posts of the first and second pairs of opposed support posts are adjustable independently of the respective positions of the second ends of the first and second transverse assemblies relative to the distal support posts of the first and second pairs of opposed support posts.

Aspect 14: The frame of aspect 13, wherein the first and second ends of the shafts of the first and second transverse support assemblies are selectively moveable relative to the vertical axis to position the central axis of the shaft of each transverse support assembly at a selected angle relative to the transverse axis, wherein the selected angle ranges from 0° to about 45°.

Aspect 15: The frame of aspect 3, further comprising a plurality of fixation assemblies that operatively couple the at least one longitudinal support assembly to the first and second transverse support assemblies, wherein each fixation assembly comprises a first receptacle configured to receive an end portion of a respective longitudinal support assembly and a second receptacle configured to receive the shaft of a respective transverse support assembly.

Aspect 16: The frame of any one of the preceding aspects, wherein at least one of the first and second transverse support assemblies further comprises at least one connector securely coupled to the shaft of the transverse support assembly and configured to engage and support a surgical tool.

Aspect 17: The frame of aspect 11, wherein at least one support post of the frame comprises at least one connector securely coupled to the support post and configured to engage and support a surgical tool.

Aspect 18: The frame of aspect 11, further comprising: a first plurality of fixation assemblies that operatively couple the at least one longitudinal support assembly to the first and second transverse support assemblies, wherein each fixation assembly of the first plurality of fixation assemblies comprises a first receptacle configured to receive an end portion of a respective longitudinal support assembly and a second receptacle configured to receive the shaft of a respective transverse support assembly; and a second plurality of fixation assemblies that operatively couple the first and second transverse support assemblies to the first and second pairs of opposed support posts, wherein each fixation assembly of the second plurality of fixation assemblies comprises a first receptacle configured to receive the shaft of a respective transverse support assembly and a second receptacle configured to receive a respective support post.

Aspect 19: A frame for supporting one or more surgical tools during a surgical procedure, the frame being connectable to a surgical table, the frame comprising: at least one support assembly, the at least one support assembly having at least one connector configured to engage and support a surgical tool, wherein the frame is connectable to the surgical table such that the at least one support assembly is spaced from the surgical table in an upward direction relative to a vertical axis, and wherein a first support assembly of the at least one support assembly is radiolucent.

Aspect 20: A method of using one or more surgical tools, comprising: connecting a frame to a surgical table, the frame comprising: first and second transverse support assemblies, each transverse support assembly extending from a proximal side of a surgical table to an opposed distal side of the surgical table and having a shaft with a central axis, wherein the second transverse support assembly is spaced from the first transverse support assembly relative to a longitudinal axis of the surgical table, wherein the proximal and distal sides of the surgical table extend between opposed first and second ends of the surgical table, and wherein the proximal side of the surgical table is spaced apart from the distal side of the surgical table relative to a transverse axis that is substantially perpendicular to the longitudinal axis of the surgical table; and at least one longitudinal support assembly extending between and operatively coupled to the first and second transverse support assemblies, wherein the at least one longitudinal support assembly comprises a shaft and at least one connector securely coupled to the shaft and configured to engage and support a surgical tool, wherein the frame is connected to the surgical table such that the first and second transverse support assemblies and the at least one longitudinal support assembly are spaced from the surgical table in an upward direction relative to a vertical axis that is substantially perpendicular to both the longitudinal axis of the surgical table and a transverse axis of the surgical table; securing at least one surgical tool to a connector of the frame; and selectively activating the at least one surgical tool.

Aspect 21: The method of aspect 20, wherein the at least one longitudinal support assembly is radiolucent.

Aspect 22: The method of aspect 21, further comprising producing an X-Ray image of a portion of a surgical field while at least a portion of the at least one longitudinal support assembly is positioned over or within the surgical field.

Aspect 23: The method of any one of aspects 20-22, wherein the at least one surgical tool comprises a surgical retractor.

Aspect 24: The method of any one of aspects 20-23, wherein each longitudinal support assembly of the at least one longitudinal support assembly has a first end portion operatively coupled to the shaft of the first transverse support assembly and a second end portion operatively coupled to the shaft of the second transverse support assembly, and wherein the method further comprises: selectively axially moving the first end portion of the longitudinal support assembly relative to the central axis of the shaft of the first transverse support assembly; and selectively axially moving the second end portion of the longitudinal support assembly relative to the central axis of the shaft of the second transverse support assembly.

Aspect 25: The method of any one of aspects 20-24, wherein the frame further comprises: at least first and second pairs of opposed support posts, each support post having a first end and an opposed second end, wherein the first end of each support post is operatively connected to the table, wherein each support post extends upwardly from the first end of the support post such that the second end of the support post is spaced from the first end of the support post relative to the vertical axis, wherein each pair of opposed support posts has a proximal support post and a distal support post that are spaced apart relative to the transverse axis of the table, wherein the proximal support post is operatively connected to the proximal side of the table and the distal support post is operatively connected to the distal side of the table, wherein the first pair of opposed support posts is operatively coupled to and supports the first transverse support assembly, and wherein the second pair of opposed support posts is operatively coupled to and supports the second transverse support assembly.

Although several embodiments of the invention have been disclosed in the foregoing specification, it is understood by those skilled in the art that many modifications and other embodiments of the invention will come to mind to which the invention pertains, having the benefit of the teaching presented in the foregoing description and associated drawings. It is thus understood that the invention is not limited to the specific embodiments disclosed hereinabove, and that many modifications and other embodiments are intended to be comprised within the scope of the appended claims. Moreover, although specific terms are employed herein, as well as in the claims which follow, they are used only in a generic and descriptive sense, and not for the purposes of limiting the described invention, nor the claims which follow.

What is claimed is:

1. A frame for supporting one or more surgical tools during a surgical procedure, the frame being connectable to a surgical table and comprising:
first and second transverse support assemblies, each transverse support assembly being configured to extend from a proximal side of a surgical table to an opposed distal side of the surgical table and having a shaft with a central axis, wherein the second transverse support assembly is spaced from the first transverse support assembly relative to a longitudinal axis of the surgical table, wherein the proximal and distal sides of the surgical table extend between opposed first and second ends of the surgical table, and wherein the proximal side of the surgical table is spaced apart from the distal side of the surgical table relative to a transverse axis that is substantially perpendicular to the longitudinal axis of the surgical table; and
at least one longitudinal support assembly extending between and operatively coupled to the first and second transverse support assemblies, wherein the at least one longitudinal support assembly comprises a shaft and at least one connector securely coupled to the shaft and configured to engage and support a surgical tool,
wherein the frame is connectable to the surgical table such that the first and second transverse support assemblies and the at least one longitudinal support assembly are spaced from the surgical table in an upward direction relative to a vertical axis that is substantially perpendicular to both the longitudinal axis of the surgical table and a transverse axis of the surgical table.

2. The frame of claim 1, wherein the at least one longitudinal support assembly is radiolucent.

3. The frame of claim 1, wherein each longitudinal support assembly of the at least one longitudinal support assembly has a first end portion operatively coupled to the shaft of the first transverse support assembly and a second end portion operatively coupled to the shaft of the second transverse support assembly, wherein the first end portion of the longitudinal support assembly is selectively axially moveable relative to the central axis of the shaft of the first transverse support assembly, and wherein the second end portion of the longitudinal support assembly is selectively axially moveable relative to the central axis of the shaft of the second transverse support assembly.

4. The frame of claim 3, wherein the shaft of each longitudinal support assembly of the at least one longitudinal support assembly has a central axis, wherein the first and second end portions of each longitudinal support assembly are selectively moveable relative to the central axes of the shafts of the first and second transverse support assemblies to position the central axis of the longitudinal support assembly at a selected angle relative to the longitudinal axis of the table, wherein the selected angle ranges from 0° to about 45°.

5. The frame of claim 3, wherein each longitudinal support assembly has a length that is adjustable to permit independent axial movement of the first and second end portions of the longitudinal support assembly relative to the respective central axes of the shafts of the first and second transverse support assemblies.

6. The frame of claim 5, wherein the first and second end portions of each longitudinal support assembly are respectively pivotally coupled to the shafts of the first and second transverse support assemblies.

7. The frame of claim 6, wherein the shaft of each longitudinal support assembly of the at least one longitudinal support assembly defines a central conduit, and wherein at least one of the first end portion and the second end portion of the longitudinal support element comprises an elongate element that is at least partially received within the central conduit of the shaft and slidably coupled to the shaft to permit adjustment of the length of the longitudinal support assembly.

8. The frame of claim 7, wherein both the first end portion and the second end portion of each longitudinal support assembly of the at least one longitudinal support assembly comprise a respective elongate element that is at least partially received within the central conduit of the shaft and slidably coupled to the shaft to permit adjustment of the length of the longitudinal support assembly.

9. The frame of claim 3, wherein both the first and second end portions of each longitudinal support assembly comprise a respective elongate element that is pivotally coupled to the shaft of the longitudinal support assembly.

10. The frame of claim 3, wherein the shafts of both the first and second transverse support assemblies are respectively adjustably coupled to the first and second end portions of each longitudinal support assembly to permit adjustment of the position of the first and second transverse support assemblies relative to the longitudinal axis of the table.

11. The frame of claim 1, wherein the frame further comprises:
at least first and second pairs of opposed support posts, each support post having a first end and an opposed second end, wherein the first end of each support post is operatively connectable to the table, wherein each support post extends upwardly from the first end of the support post such that the second end of the support post is spaced from the first end of the support post relative to the vertical axis, wherein each pair of opposed support posts has a proximal support post and a distal support post that are spaced apart relative to the transverse axis of the table, wherein the proximal support post is operatively connectable to the proximal side of the table and the distal support post is operatively connectable to the distal side of the table, wherein the first pair of opposed support posts is operatively coupled to and configured to support the first transverse support assembly, and wherein the second pair of opposed support posts is operatively coupled to and configured to support the second transverse support assembly.

12. The frame of claim 11, wherein the shafts of both the first and second transverse support assemblies are respectively adjustably coupled to the first and second pairs of opposed support posts to permit adjustment of the position of the first and second transverse support assemblies relative to the vertical axis.

13. The frame of claim 12, wherein the shafts of both the first and second transverse support assemblies have opposed first and second ends, wherein the first ends of the shafts of the first and second transverse support assemblies are respectively adjustably coupled to the proximal support posts of the first and second pairs of opposed support posts, wherein the second ends of the shafts of the first and second transverse support assemblies are respectively adjustably coupled to the distal support posts of the first and second pairs of opposed support posts, and wherein the respective positions of the first ends of the first and second transverse support assemblies relative to the proximal support posts of the first and second pairs of opposed support posts are adjustable independently of the respective positions of the second ends of the first and second transverse assemblies relative to the distal support posts of the first and second pairs of opposed support posts.

14. The frame of claim 13, wherein the first and second ends of the shafts of the first and second transverse support assemblies are selectively moveable relative to the vertical axis to position the central axis of the shaft of each transverse support assembly at a selected angle relative to the transverse axis, wherein the selected angle ranges from 0° to about 45°.

15. The frame of claim 3, further comprising a plurality of fixation assemblies that operatively couple the at least one longitudinal support assembly to the first and second transverse support assemblies, wherein each fixation assembly comprises a first receptacle configured to receive an end portion of a respective longitudinal support assembly and a second receptacle configured to receive the shaft of a respective transverse support assembly.

16. The frame of claim 1, wherein at least one of the first and second transverse support assemblies further comprises at least one connector securely coupled to the shaft of the transverse support assembly and configured to engage and support a surgical tool.

17. The frame of claim 11, wherein at least one support post of the frame comprises at least one connector securely coupled to the support post and configured to engage and support a surgical tool.

18. The frame of claim 11, further comprising:
a first plurality of fixation assemblies that operatively couple the at least one longitudinal support assembly to the first and second transverse support assemblies, wherein each fixation assembly of the first plurality of fixation assemblies comprises a first receptacle configured to receive an end portion of a respective longitudinal support assembly and a second receptacle configured to receive the shaft of a respective transverse support assembly; and
a second plurality of fixation assemblies that operatively couple the first and second transverse support assemblies to the first and second pairs of opposed support posts, wherein each fixation assembly of the second plurality of fixation assemblies comprises a first receptacle configured to receive the shaft of a respective transverse support assembly and a second receptacle configured to receive a respective support post.

19. A frame for supporting one or more surgical tools during a surgical procedure, the frame being connectable to a surgical table, the frame comprising:
at least one support assembly, the at least one support assembly having at least one connector configured to engage and support a surgical tool,
wherein the frame is connectable to the surgical table such that the at least one support assembly is spaced from the surgical table in an upward direction relative to a vertical axis, and
wherein a first support assembly of the at least one support assembly is radiolucent.

20. A method of using one or more surgical tools, comprising:
connecting a frame to a surgical table, the frame comprising:
first and second transverse support assemblies, each transverse support assembly extending from a proximal side of a surgical table to an opposed distal side of the surgical table and having a shaft with a central axis, wherein the second transverse support assembly is spaced from the first transverse support assembly relative to a longitudinal axis of the surgical table, wherein the proximal and distal sides of the surgical table extend between opposed first and second ends of the surgical table, and wherein the proximal side of the surgical table is spaced apart from the distal side of the surgical table relative to a transverse axis that is substantially perpendicular to the longitudinal axis of the surgical table; and
at least one longitudinal support assembly extending between and operatively coupled to the first and second transverse support assemblies, wherein the at least one longitudinal support assembly comprises a shaft and at least one connector securely coupled to the shaft and configured to engage and support a surgical tool,
wherein the frame is connected to the surgical table such that the first and second transverse support assemblies and the at least one longitudinal support assembly are spaced from the surgical table in an upward direction relative to a vertical axis that is substantially perpendicular to both the longitudinal axis of the surgical table and a transverse axis of the surgical table;
securing at least one surgical tool to a connector of the frame; and
selectively activating the at least one surgical tool.
